(19) United States
(12) Patent Application Publication
(10) Pub. No.: US 2021/0378644 A1
(43) Pub. Date: Dec. 9, 2021

(54) DEVICE AND METHODS FOR TRANSRECTAL ULTRASOUND-GUIDED PROSTATE BIOPSY

(71) Applicant: THE JOHNS HOPKINS UNIVERSITY, Baltimore, MD (US)

(72) Inventors: Dan Stoianovici, Reisterstown, MD (US); Sunghwan Lim, Boan-myeon (KR); Misop Han, West Friendship, MD (US)

(21) Appl. No.: 17/289,128

(22) PCT Filed: Dec. 3, 2019
(86) PCT No.: PCT/US2019/064208

§ 371 Date: Apr. 27, 2021

Related U.S. Application Data
(60) Provisional application No. 62/774,559, filed on Dec. 3, 2018.

(51) Int. Cl. A61B 10/02; A61B 34/20; A61B 34/30; A61B 8/12; A61B 8/00; A61B 8/08

(52) U.S. Cl. CPC A61B 10/0241; A61B 34/20; A61B 34/30; A61B 2034/2063; A61B 8/4218; A61B 8/0841; A61B 8/12

(57) ABSTRACT

A robot-assisted approach for transrectal ultrasound (TRUS) guided prostate biopsy includes a hands-free probe manipulator that moves the probe with the same 4 degrees-of-freedom (DoF) that are used manually. Transrectal prostate biopsy is taken one step further, with an actuated TRUS manipulation arm. The robot of the present invention enables the performance of hands-free, skill-independent prostate biopsy. Methods to minimize the deformation of the prostate caused by the probe at 3D imaging and needle targeting are included to reduce biopsy targeting errors. The present invention also includes a prostate coordinate system (PCS). The PCS helps defining a systematic biopsy plan without the need for prostate segmentation. A novel method to define an SB plan is included for 3D imaging, biopsy planning, robot control, and navigation.

### CROSS REFERENCE TO RELATED APPLICATIONS

This application claims the benefit of U.S. Provisional Patent Application No. 62/774,559 filed on Dec. 3, 2018, which is incorporated by reference, herein, in its entirety.

### FIELD OF THE INVENTION

The present invention relates generally to biopsy. More particularly the present invention relates to a device and methods for transrectal, ultrasound-guided prostate biopsy.

### BACKGROUND OF THE INVENTION

Prostate cancer (PCa) is the most common non-cutaneous malignancy and the second leading cause of cancer related death among US men. Nearly 1 of every 6 men will be diagnosed with the disease at some time in their lives. The best current estimate of PCa aggressiveness is the Gleason score obtained from core needle biopsy. The most common biopsy method is freehand transrectal ultrasound (TRUS) guided. Since ultrasound only rarely identifies PCa visually, systematic biopsy (SB) intends to sample the prostate evenly. But freehand biopsy is highly inconsistent, subjective, and results in uneven sampling, leaving large regions of the prostate unsampled, which can lead to under-sampling of clinically significant PCa, and implicitly under-staging of PCa diagnosis. In response, the current trend is directed towards a targeted biopsy (TB) approach guided by multiparametric Magnetic Resonance Imaging (mpMRI). TB has advantages over SB because it allows the biopsy needle to be guided to sampling areas based on imaging that shows cancer suspicious regions (CSR). TB methods include direct in-bore MRI targeting and methods that register (fuse) pre-acquired MRI to interventional ultrasound: cognitive fusion and device/software aided fusion. Current fusion biopsy devices include: Artemis (Eigen), PercuNav (Philips), UroNav (Invivo), UroStation (Koelis), and BK Ultrasound systems.

With the fusion, few cores directed towards the CSRs are taken in addition to the 12-cores of SB. TB cores yield a higher cancer detection rate of clinically significant PCa than SB cores. But TB cores miss a large number of clinically significant PCa detected by SB, because mpMRI itself has 5%-15% false-negative clinically significant cancer detection rate. A recent multicenter randomized trial allowed men with normal mpMRI (PI-RADS≤2) to avoid biopsy and reported that TB alone may be preferable to the routine freehand SB. But the study does not tell how many men in whom biopsy was not performed might harbor clinically significant PCa. TB alone is risky and SB plays an important role in prostate diagnosis: 1) Fusion can only be offered to patients with mpMRI findings, yet 21% of biopsy patients have none (range 15%-30%, 3544 patients). SB on patients with no mpMRI findings found 42% of men to harbor PCa, of which ⅓ were clinically significant PCa; 2) On equivocal mpMRI lesions (PI-RADS=3), TB alone misses 56% of Gleason 7-10 cancers; 3) The MRI for TB adds $700-$1,500/case, and reliable mpMRI interpretation is limited. The large majority of over 1 million prostate biopsies performed annually in the US are SB. Therefore, SB plays an important role independently and together with TB.

Commonly, SB and TB are freehand procedures performed under transrectal ultrasound guidance with the TRUS probe manually operated by a urologist and a needle passed alongside the probe. To acquire ultrasound images, the TRUS probe must maintain contact with the rectal wall for the sonic waves to propagate, in turn pushing against the prostate. The TRUS probe is known to deform the gland, and the amount of pressure is typically variable throughout the procedure. Images at different regions of the prostate use different compression. If the deformed 2D images are rendered in 3D, the actual shape and volume of the gland are skewed. Further, if a biopsy plan (SB or TB) is made on the skewed images, the plan is geometrically inaccurate. Moreover, when the needle is inserted for biopsy, the probe deforms the prostate differently contributing to additional targeting errors. The errors can be significant, for example 2.35 to 10.1 mm (mean of 6.11 mm). Ideally, targeting errors for PCa biopsy should be <5 mm (clinically significant PCa lesion ≥5 cm³ in volume).

Biopsy planning and needle targeting errors are problematic for both SB and TB. At fusion TB, pre-acquired mpMRI is registered to the interventional TRUS images. The registration is typically performed by aligning the shapes of the gland in ultrasound and MRI. This alignment is challenging due to shape differences caused by the dissimilar timing, patient positioning, imaging modalities, etc. Prostate deformations by the TRUS probe further magnify the registration problem. Several elastic registration algorithms have been developed to reduce errors, and improved the initial registration. However, handling prostate deformations at the time of each needle insertion for biopsy remains problematic.

Reducing prostate deformations at biopsy has been achieved on the transperineal needle path, for example with the TargetScan device and Mona Lisa robot. However, no current transrectal biopsy device can reliably minimize prostate deformations. Most devices freehand the probe and inherently deform the prostate unevenly. The only device that offers probe handling assistance is the Artemis device, which uses a mechanical encoded TRUS support arm. This arm helps to reduce deformations, but its manual operation leads to variability among urologists.

It would therefore be advantageous to provide a device and method that allows for hands-free TRUS guided prostate biopsy that reduces prostate deformation and is consistent between urologists.

### SUMMARY

According to a first aspect of the present invention a system for prostate biopsy includes a robot-operated, hands-free TRUS-ultrasound probe and manipulation arm. The system includes a biopsy needle. The system also includes a robot controller. The robot controller is configured to communicate with and control the manipulation arm and TRUS-ultrasound probe in a manner that minimizes prostate deflection. The system also includes an ultrasound module for viewing images from the TRUS-ultrasound probe.

In accordance with an aspect of the present invention, the system further includes the robot controller being programmed with a prostate coordinate system. The robot controller is programmed with a systematic biopsy plan. The robot controller allows for computer control of the TRUS-ultrasound probe and manipulation arm. The robot controller allows for physician control of the TRUS-ultrasound probe and manipulation arm. The manipulation arm moves the probe with 4-degrees-of-freedom.

In accordance with another aspect of the present invention, the prostate control system includes a program for determining the prostate coordinate system based on anatomical landmarks of the prostate. The anatomical landmarks are the apex (A) and base (B) of the prostate. The program for determining the prostate coordinate system further includes using A and B to determine a prostate coordinate system (PCS) for the prostate. The program also includes determining the direction of the PCS based on the Left-Posterior-Superior (LPS) system, wherein an S axis is aligned along the AB direction and P is aligned with a saggital plane. The system includes calculating an optimal approach and order for a set of biopsy points determined from the PCS.

In accordance with still another aspect of the present invention, the robot controller is programmed with a systematic or targeted biopsy plan. The robot controller allows for computer control of the ultrasound probe and manipulation arm. The robot controller allows for physician control of the ultrasound probe and manipulation arm. The manipulation arm moves the probe with 4-degrees-of-freedom.

In accordance with yet another aspect of the present invention, the system includes a microphone, wherein the microphone triggers automatic acquisition of ultrasound images based on firing noise or a signal from the biopsy needle. The ultrasound probe is configured to apply minimal pressure over a prostate gland to avoid prostate deformations and skewed imaging. The prostate can be approached with minimal pressure and deformations also for biopsy. The system includes automatically acquiring images from medical imaging equipment based on firing noise of a biopsy needle, or signal from another medical instrument. The images are acquired for a purpose of documenting a clinical measure.

In accordance with another aspect of the present invention, a method for biopsy of a prostate includes determining a midpoint between an apex (A) and base (B) of the prostate. The method also includes using A and B to determine a prostate coordinate system (PCS) for the prostate and determining the direction of the PCS based on the Left-Posterior-Superior (LPS) system, wherein an S axis is aligned along the AB direction and P is aligned with a saggital plane. The method includes calculating an optimal approach and order for a set of biopsy points determined from the PCS.

In accordance with even another aspect of the present invention, the method includes imaging the prostate with an ultrasound probe with minimal pressure over a prostate gland to avoid prostate deformations and skewed imaging. The prostate can be approached with minimal pressure and deformations also for biopsy. The method includes automatically acquiring images from medical imaging equipment based on firing noise of a biopsy needle, or signal from another medical instrument. The method includes acquiring the images for a purpose of documenting a clinical measure. The method also includes triggering automatic acquisition of ultrasound images based on firing noise or a signal from the biopsy needle acquired by a microphone. Additionally, the method includes computer control of the ultrasound probe and manipulation arm. The computer control allows for physician control of the ultrasound probe and manipulation arm.

### BRIEF DESCRIPTION OF THE DRAWINGS

The accompanying drawings provide visual representations which will be used to more fully describe the representative embodiments disclosed herein and can be used by those skilled in the art to better understand them and their inherent advantages. In these drawings, like reference numerals identify corresponding elements and:

FIG. 1 illustrates a side view of a robot manipulator having an RCM module and RT driver.

FIG. 2 illustrates a schematic diagram of the TRUS-guided robotics prostate biopsy system of the present invention.

FIGS. 3A-3C illustrate views of the graphic user interface (GUI) with three main components: robot control, as illustrated in FIG. 3A; virtual reality for biopsy planning including real-time robot positioning, 3D ultrasound image and biopsy plan, as illustrated in FIG. 3B; and navigation screen showing real-time ultrasound and green guide line showing the direction of the biopsy needle and insertion depth before firing the biopsy, so that after firing the core is centered at the target, as illustrated in FIG. 3C.

FIGS. 4A and 4B illustrate perspective views of an ultrasound probe and calibration of the ultrasound probe.

FIGS. 5A and 5B illustrate inverse kinematics of the robot manipulator.

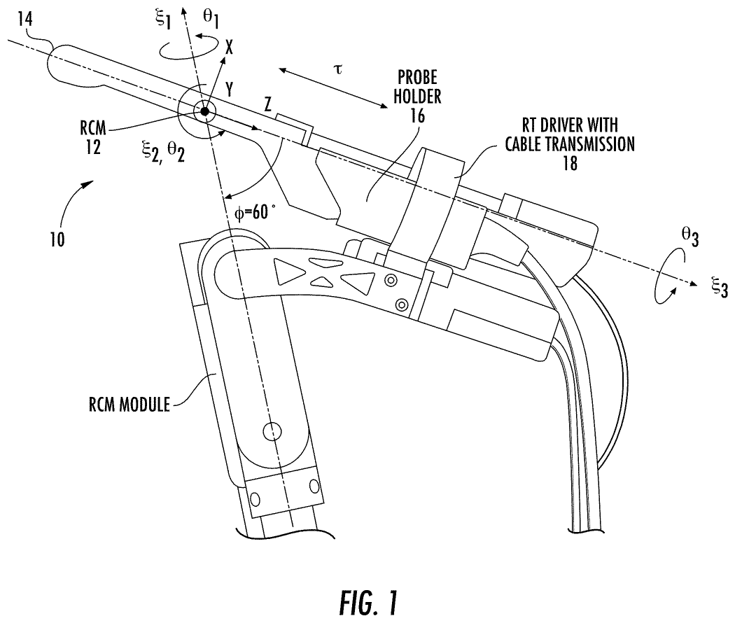

FIG. 1 illustrates a graphical view of an example of optimizing the approach angles for target point p=(10, 10, −100)^{T} and scan position (θ₁^{s}, θ₂^{s})=(0, 0).

FIGS. 7A and 7B illustrate graphical views of examples of the location of 12 biopsy cores in joint coordinates, as illustrated in FIG. 7A and Cartesian coordinates, as illustrated in FIG. 7B.

FIGS. 8A-8D illustrate image views of prostate biopsy plans.

FIG. 9 illustrates a perspective view of an experimental setup for robot joint accuracy test.

FIGS. 10A-10C illustrate the 3D Imaging Geometric Accuracy Test and the Grid Targeting Test.

FIGS. 11A and 11B illustrate a targeting experiment with prostate mock-up.

FIGS. 12A and 12B illustrate schematic diagrams of prostate displacement and prostate deformation measurements, respectively.

FIG. 13 illustrates a side view of a robotic prostate biopsy.

FIG. 14 illustrates a graphical view of the Robot set point test results (θ₃=0°).

FIGS. 15A and 15B illustrate image views of targeting results with prostate mock-up.

FIG. 16A illustrates common handing the probe to a site, and FIG. 16B illustrates an optimal handing the probe to a site.

### DETAILED DESCRIPTION OF THE PREFERRED EMBODIMENTS

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

A robot-assisted approach for transrectal ultrasound (TRUS) guided prostate biopsy includes a hands-free probe manipulator that moves the probe with the same 4 degrees-of-freedom (DoF) that are used manually. Transrectal prostate biopsy is taken one step further, with an actuated TRUS manipulation arm. The robot of the present invention enables the performance of hands-free, skill-independent prostate biopsy. Methods to minimize the deformation of the prostate caused by the probe at 3D imaging and needle targeting are included to reduce biopsy targeting errors. The present invention also includes a prostate coordinate system (PCS). The PCS helps defining a systematic biopsy plan without the need for prostate segmentation. A novel method to define an SB plan is included for 3D imaging, biopsy planning, robot control, and navigation.

Comprehensive tests were performed, including 2 bench tests, 1 imaging test, 2 in vitro targeting tests, and an IRB-approved clinical trial on 5 patients. Preclinical tests showed that image-based needle targeting can be accomplished with accuracy on the order of 1 mm. Prostate biopsy can be accomplished with minimal TRUS pressure on the gland and submillimetric prostate deformations. All 5 clinical cases were successful with an average procedure time of 13 min and millimeter targeting accuracy. Hands-free TRUS operation, transrectal TRUS guided prostate biopsy with minimal prostate deformations, and the PCS based biopsy plan are novel methods. Robot-assisted prostate biopsy is safe and feasible. Accurate needle targeting has the potential to increase the detection of clinically significant prostate cancer.

A robot according to the present invention is a TRUS probe manipulator that moves the probe with the same 4 degrees-of-freedom (DoF) that are used manually in transrectal procedures, closely replicating its movement by hand, but eliminating prostate deformation and variation between urologists. FIG. 1 illustrates a side view of a robot manipulator having an RCM module and RT driver. The TRUS probe 10 can pivot in two directions (ξ₁ and ξ₂) about a fulcrum point (RCM) 12 that is to be located at the anus, can be inserted or retracted (along axis ξ₃), and spun about its axis (ξ₃). The rotations about the fulcrum point are performed with a Remote Center of Motion (RCM) mechanism 12. The RCM 12 of the present invention is relatively small and uses belts to implement the virtual parallelogram.

For biopsy, the robot includes a backlash-free cable transmission for the ξ₃ rotary axis and (previous used gears), and larger translational range along the ξ₃ axis. The hardware limits of the joints in a preferred embodiment are: θ₁ about ξ₁(±86°), θ₂ about ξ₂ (−17° to 46°), θ₃ about δ₃(±98°, τ along ξ₃(±49 mm).

The robot is supported by a passive arm which mounts on the side of the procedure table. With special adapters, the robot can support various probes. A 2D end-fire ultrasound probe (EUP-V53W, Hitachi Medical Corporation, Japan) was mounted in the robot and connected to a Hitachi HI VISION Preirus machine. As shown in FIG. 1, the probe 10 is mounted so that axis ξ₃ is centered over the semi-spherical shaped point 14 of the probe 10. As illustrated in FIG. 1, the probe 10 is generally a TRUS probe disposed in a probe holder 16. The probe holder 16 is coupled to an RT driver 18. The RT driver 18 has cable transmission. The RT driver is in turn coupled to the RCM module 12.

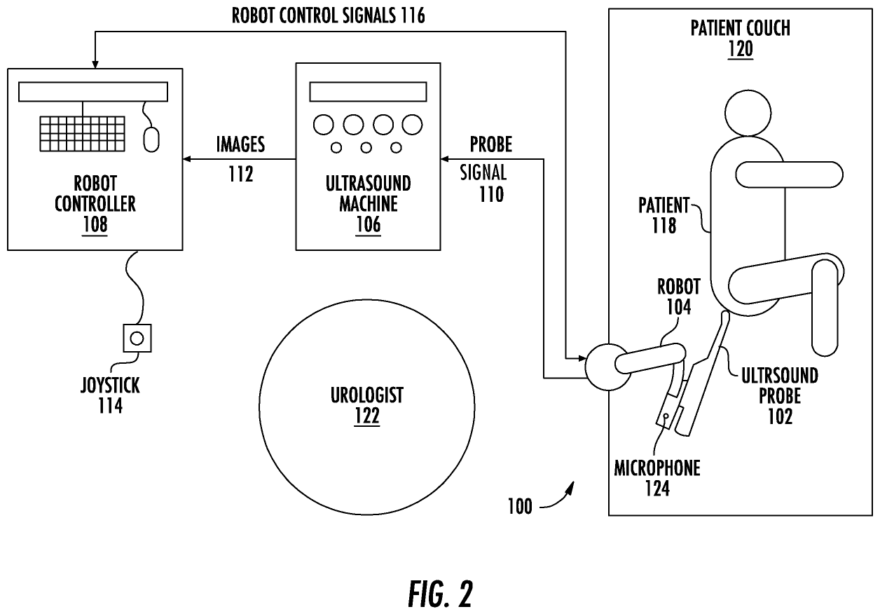

A system diagram is shown in FIG. 2. FIG. 2 illustrates a schematic diagram of the TRUS-guided robotics prostate biopsy system of the present invention. The system 100 includes the TRUS probe 102 and associated robot 104, an ultrasound device 106, and a robot controller 108. The TRUS probe 102 communicates a probe signal 110 to the ultrasound device 106, which, in turn, transmits image data 112 to the robot controller 108. A joystick 114 or other suitable controller known to or conceivable to one of skill in the art can be included. The robot controller 108 transmits robot control signals 116 to the robot 104 associated with the TRUS probe 102. The patient 118 is disposed on the patient couch 120, while the procedure is performed by urologist 122. A microphone 124 is mounted on the robot 104, in close proximity of the needle. This microphone 124 listens for the noise of the biopsy needle firing. The circuit triggers the acquisition of images form the ultrasound 106, to automatically recording the ultrasound of the image at the exact moment of biopsy sampling.

An exemplary robot controller is built with a PC with Intel® Core™ i7 3.07-GHz CPU, 8 GB RAM, NVIDIA GeForce GTX 970 GPU, Matrox Orion HD video capture board, MC8000 (PMDi, Victoria, BC, Canada) motion control board, 12V/4.25Ah UPS, and 24V power supplies. Custom software was developed in Visual C++ (Microsoft, Seattle, Wash.) using commercial libraries comprising MFC, MCI, and MIL, and open-source libraries comprising Eigen, OpenCV, OpenMP, GDCM, VTK, and ITK.

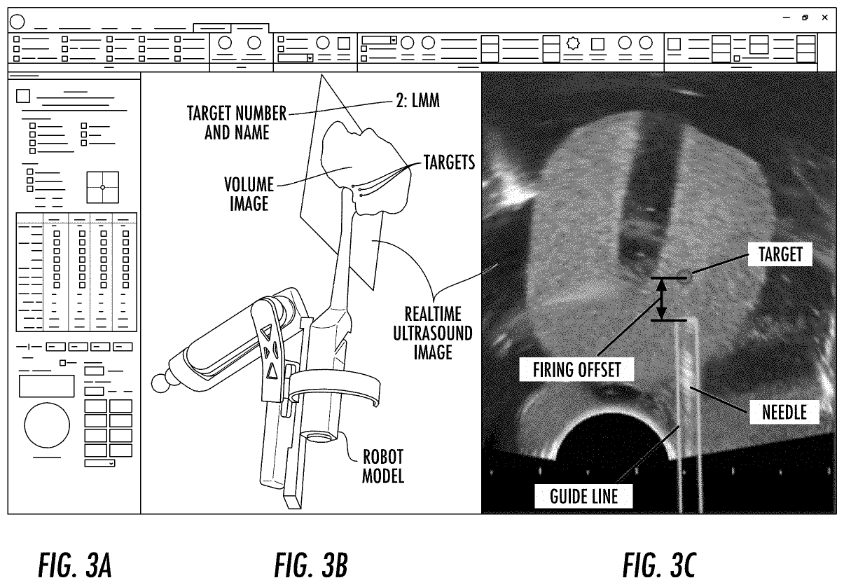

FIGS. 3A-3C illustrate views of the graphic user interface (GUI) with three main components: robot control, as illustrated in FIG. 3A; virtual reality for biopsy planning including real-time robot positioning, 3D ultrasound image and biopsy plan, as illustrated in FIG. 3B; and navigation screen showing real-time ultrasound and green guide line showing the direction of the biopsy needle and insertion depth before firing the biopsy, so that after firing the core is centered at the target, as illustrated in FIG. 3C.

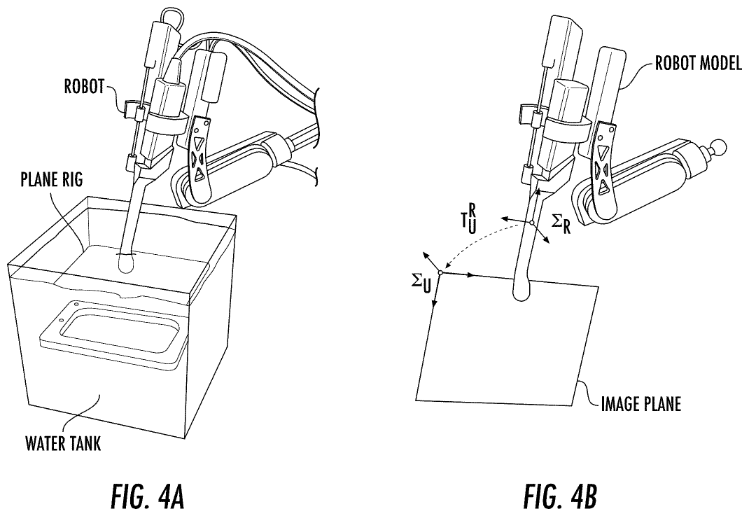

3D ultrasound is acquired from a 2D probe with a robotic scan. A one-time calibration process is required, to determine the transformation and scaling T_{U}^{R} (4×4 matrix) from the robot coordinate system Σ_{R} to the image frame Σ_{U}, as illustrated in FIGS. 4A and 4B. FIGS. 4A and 4B illustrate perspective views of an ultrasound probe and calibration of the ultrasound probe. FIG. 4A illustrates a perspective view of a setup for the ultrasound probe calibration, and FIG. 4B illustrates a schematic diagram of ultrasound probe calibration. A calibration rig is made of a thin planar plastic sheet submersed in a water tank, as illustrated in FIG. 4A. In ultrasound this appears as a line, and was automatically detected using a RANSAC algorithm at different poses of the probe set by the robot. The calibration matrix was then estimated by solving least-square problems. The process was repeated at five depth settings of the ultrasound machine (50, 65, 85, 110, and 125 mm), to have the proper calibration if the machine depth is changed.

3D ultrasound is acquired with a robotic rotary scan about ξ₃ axis. During the scan, images are acquired from the ultrasound machine over the video capture board. At the time of each image acquisition, the computer also records the current robot joint coordinates and calculates the position of the respective image frame in robot coordinates (Σ_{R}) through the calibration and forward kinematics. Overall, the raw data is a series of image-position pairs. A 3D volume image is then constructed from the raw data using a variation of Trobaugh's method. Rather than filling voxels with the mean of two pixels that are closest to the voxel regardless of distance (needed to fill all voxels in the case of a manual scan), only the pixels that are within a given distance (enabled by the uniform robotic scan) were used. The distance was set to half of the acoustic beam width (D), which is determined at calibration. The speed of the rotary scan, V_{scan}, is calculated to fill the voxels that are farthest from ξ₃, at radius R, as:

$${V}_{s c a n} = \frac{Df}{R} [ rad / s ] \qquad ( 1 )$$

where ƒ [fps] is the ultrasound frame rate (read on the machine display). Due to the rotary scan, pixels that are closer to the axis are denser, so the number of pixels that were averaged in each voxel was limited (i.e. 5). Practically, the speed of the scan is limited by the frame rate of the ultrasound machine (i.e. 15 fps).

Experimentally, the ultrasound array was not perfectly aligned with the shaft of the ultrasound probe and respectively with ξ₃. The rotary scan left blank voxels near the axis. To fill these, a small ξ₂ (3°) motion normal to the image plane was performed before the pure rotary scan.

At the time of the scan, the end-fire probe is initially set to be near the central sagittal image of the gland and the current joint values of θ₁ and θ₂ are saved as a scan position (θ₁^{s} and θ₂^{s}). The probe is then retracted (translation τ along ξ₃, typically under joystick control) until the quality of the image starts to deteriorate by losing contact, and is then slightly advanced to recover image quality. This insertion level sets the minimal pressure needed for imaging. The rotary scan is performed without changing the insertion depth. As such, the probe pressure over the gland is maintained to the minimum level throughout the scan since the axis of rotation coincides with the axis of the semi-spherical probe end and gel lubrication is used to reduce friction. The method enables 3D imaging with quasi-uniform, minimal prostate deformations. The method of the present invention below will show that the minimal deformation can also be preserved at biopsy.

For the accuracy of needle targeting according to and based on the acquired 3D image, it is essential that the gland maintains the same shape at biopsy. Therefore, the same level of prostate compression should be used as much as possible. The following 3 steps are used:

1) Optimizing the Probe Approach to Each Biopsy Site

The probe insertion level used at scanning is preserved (r is locked). Still, infinitely many solutions for the joint angles θ₁, θ₂, and θ₃ exist to approach the same target point. This is fortunate, because it leaves room to optimize the approach angles in order to minimize prostate deformations. As shown above, the rotation about the probe axis (ξ₃) preserves prostate deformations due to the semi-spherical probe point. As such, needle targeting should be performed as much as possible with ξ₃, and motions in the RCM axes ξ₁ and ξ₂, which are lateral to the probe, should be reduced. If a biopsy target point is selected in the 3D ultrasound image, the robot should automatically orient the probe so that the needle-guide points towards the target. The volume image is in robot coordinates, therefore, the target point is already in robot coordinates. Robot's inverse kinematics is required to determine the corresponding joint coordinates. Here, the specific inverse kinematics are shown that includes the needle and solves the joint angles θ₁, θ₂ for a given target point \vec{p}∈ [symbol] ³, insertion level τ, and joint angle θ₃.

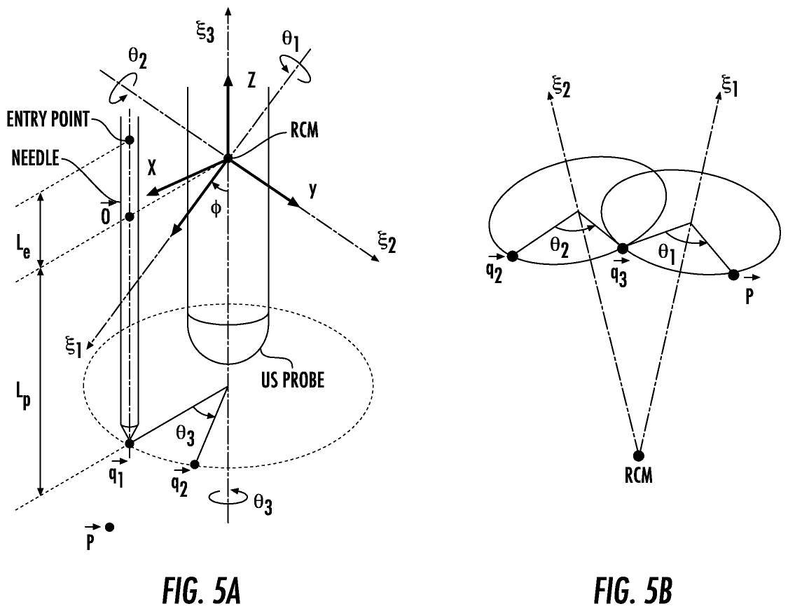

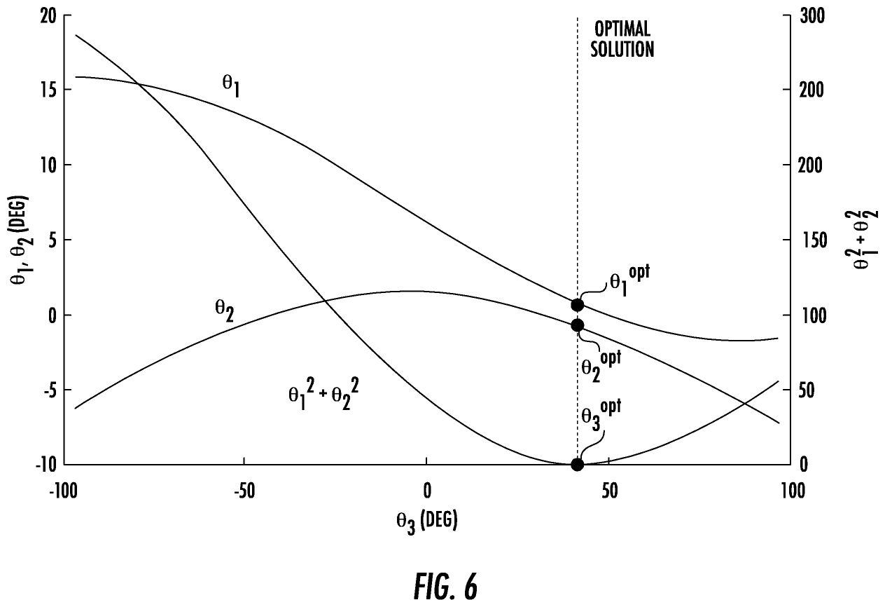

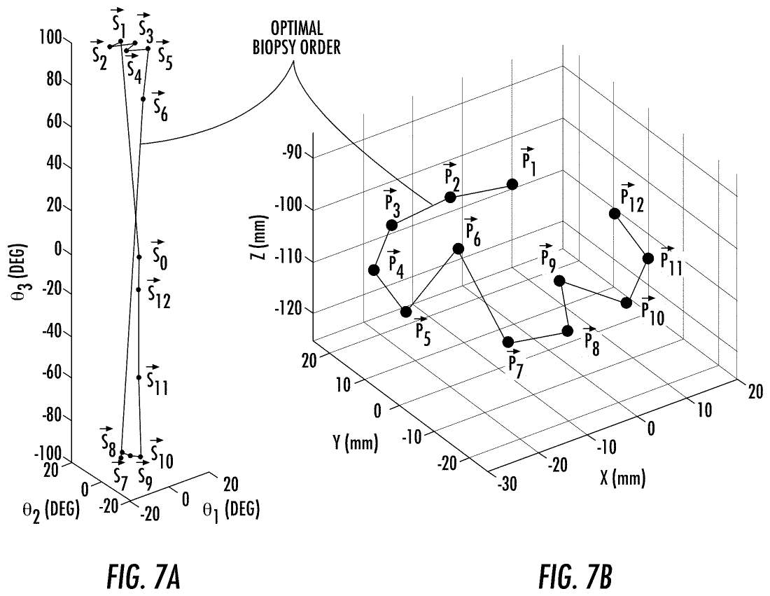

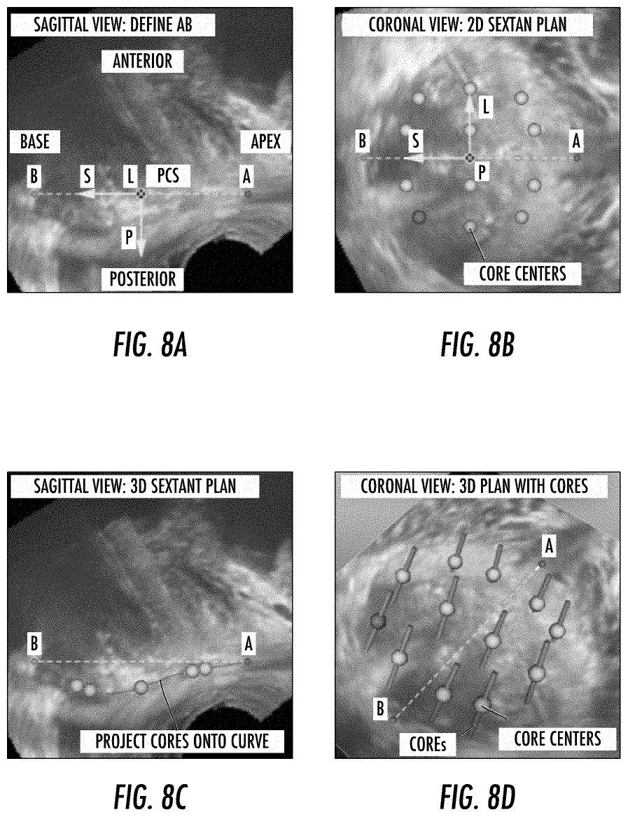

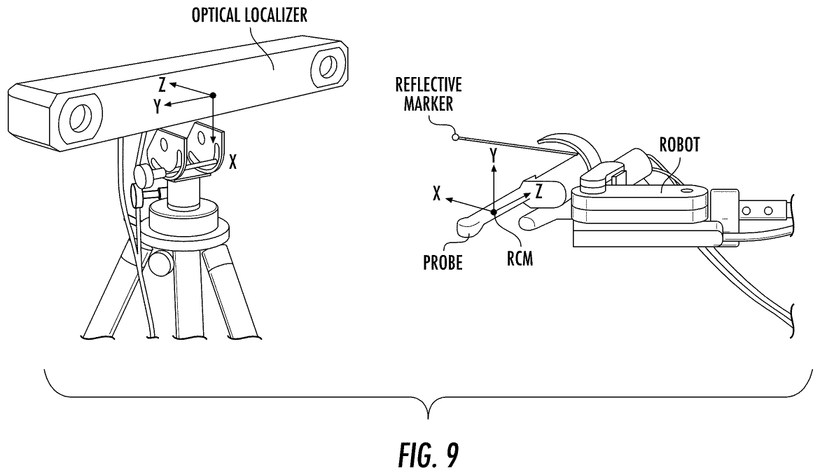

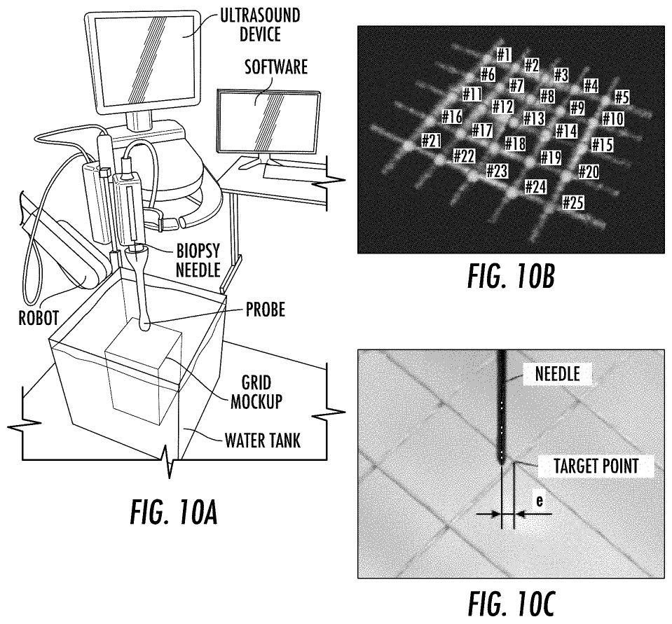

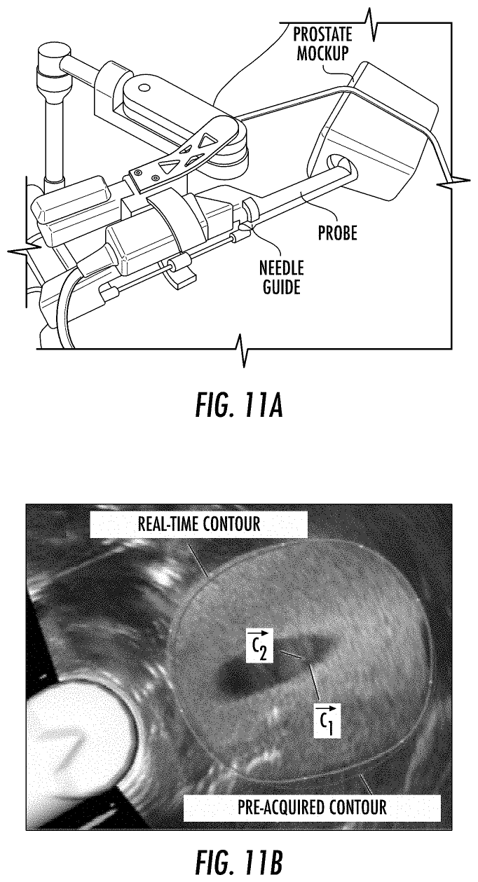

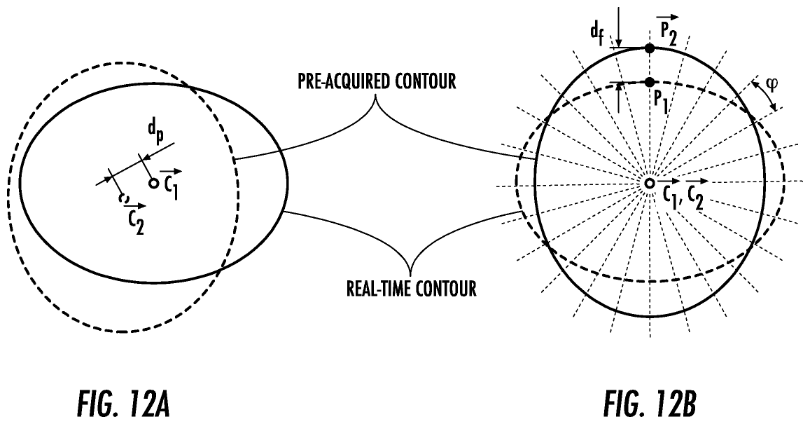

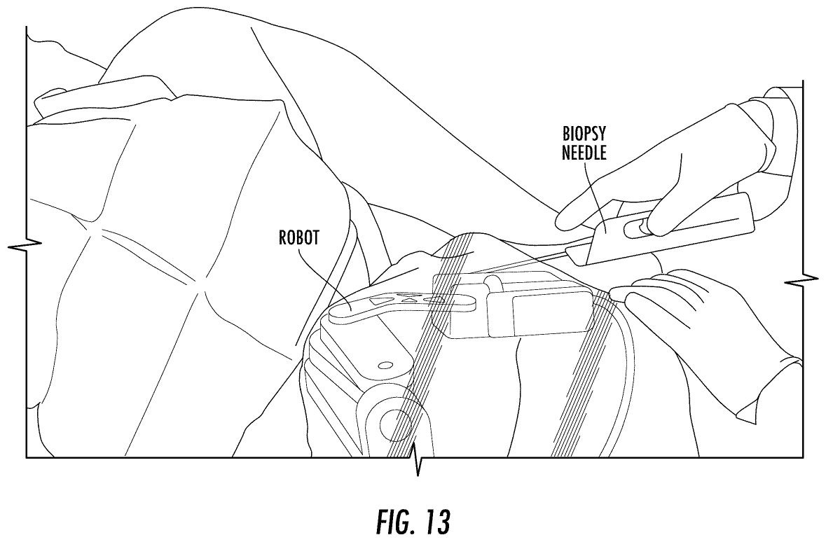

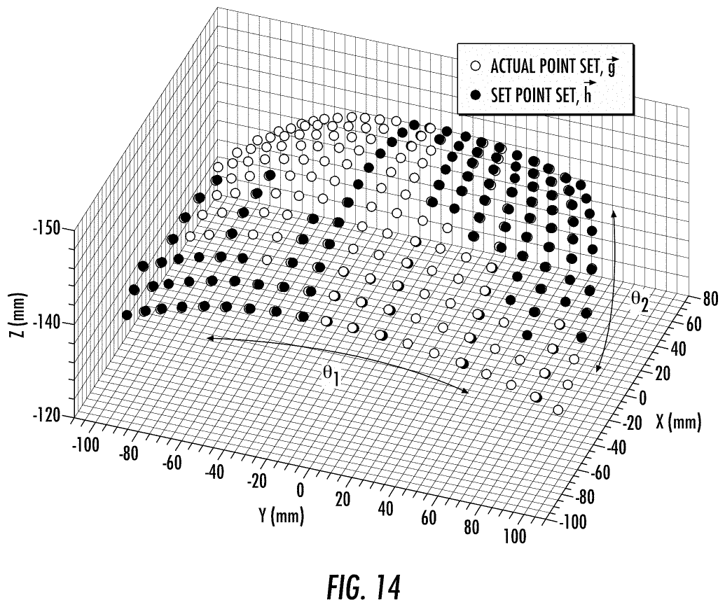

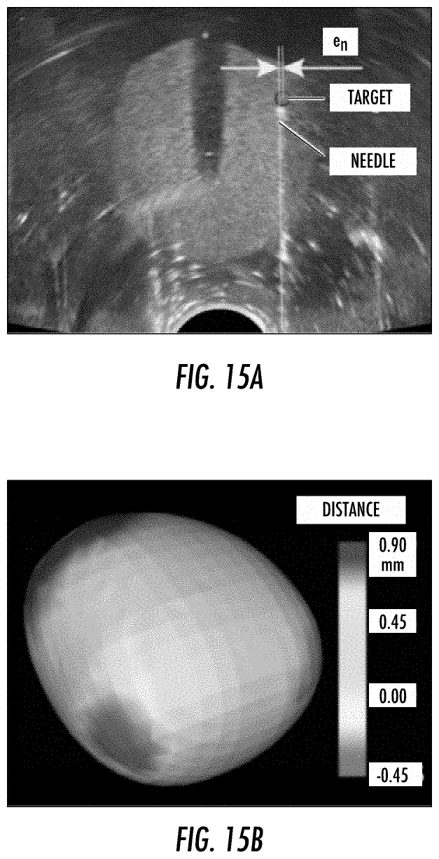

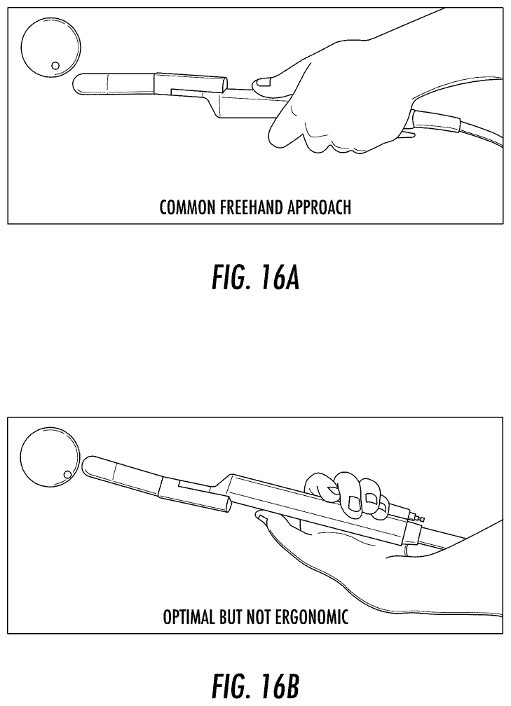

FIGS. 5A and 5B illustrate inverse kinematics of the robot manipulator. FIG. 5A illustrates inverse kinematics for a given target point p and rotation angle θ₃, and FIG. 5B illustrates inverse kinematics to find the rotation angles θ₁ and θ₂.

As shown in FIGS. 5A and 5B the needle-guide passes through a point \vec{o}=(oₓ, o_{y}, 0)^{T} (known from design and calibration) and is parallel to ξ₃. For the target point \vec{p} and chosen θ₃, joint angles θ₁ and θ₂ have unique solutions, calculated with the second Paden-Kahan sub-problem approach, as follows.

The axes of the robot are:

ξ₁=(sinϕ,0,−cosϕ)^{T}

ξ₂=(0,1,0)^{T} (1)

ξ₃=(0,0,1)^{T}

where ϕ=60° is a constant offset angle. The needle insertion depth L required to place the needle point at the target \vec{p} is:

L=Lₑ+Lₚ+τ (2)

where Lₑ is a constant distance between the entry point of the needle guide and the RCM point in the direction of the axis ξ₃, and Lₚ is a distance between the RCM point and the target point \vec{p} in the direction of the axis ξ₃ such that:

Lₚ=\sqrt{\vec{p}^{T}\vec{p}−\vec{o}^{T}\vec{o}} (3)

When the robot is in zero position as shown in FIG. 5A, the needle point \vec{q}₁ is given by:

\vec{q}₁=(oₓ,o_{y},−Lₚ)^{T} (4)

and when rotated by θ₃ is:

\vec{q}₂=e^{\hat{ξ}}^{₃}^{θ}^{₃}\vec{q}₁ (5)

where \hat{ξ}₃ is the cross-product matrix of ξ₃.

Then, θ₁ and θ₂ satisfy:

e^{\hat{ξ}}^{₁}^{θ}^{₁}e^{\hat{ξ}}^{₂}^{θ}^{₂}\vec{q}₂=\vec{p} (6)

where \hat{ξ}₁ and \hat{ξ}₂ are the cross-product matrices of ξ₁ and ξ₂, respectively. If \vec{q}₃ is a point such that:

\vec{q}₃=e^{\hat{ξ}}^{₂}^{θ}^{₂}\vec{q}₂=e^{-\hat{ξ}}^{₁}^{θ}^{₁}\vec{p} (7)

then:

\vec{q}₃=αξ₁+βξ₂+γ(ξ₁×ξ₂) (8)

where:

$$α = \frac{( {ξ}_{1}^{T} {ξ}_{2} ) {ξ}_{2}^{T} {\vec{q}}_{2} - {ξ}_{1}^{T} \vec{p}}{{( {ξ}_{1}^{T} {ξ}_{2} )}^{2} - 1} \\ β = \frac{( {ξ}_{1}^{T} {ξ}_{2} ) {ξ}_{1}^{T} \vec{p} - {ξ}_{2}^{T} {\vec{q}}_{2}}{{( {ξ}_{1}^{T} {ξ}_{2} )}^{2} - 1} \\ γ = \pm \sqrt{\frac{{\vec{q}}_{2}^{T} {\vec{q}}_{2} - {α}^{2} - {β}^{2} - 2 {αβξ}_{1}^{T} {ξ}_{2}}{{( {ξ}_{1} \times {ξ}_{2} )}^{T} ( {ξ}_{1} \times {ξ}_{2} )}} \qquad ( 9 )$$

Finally, θ₁ and θ₂ can be found by solving:

e^{\hat{ξ}}^{₂}^{θ}^{₂}\vec{q}₂=\vec{q}₃ and e^{-ξ}^{₁}^{θ}^{₁}\vec{p}=\vec{q}₃ (10)

as:

θ₂=a tan2(ξ₂^{T}(\vec{q}′₂×\vec{q}′₃),q′₂^{T}\vec{q}′₃) (10)

\vec{q}′₂=\vec{q}₂−ξ₂ξ₂^{T}\vec{q}₂

\vec{q}′₃=\vec{q}₃−ξ₂ξ₂^{T}\vec{q}₃

θ₁=−a tan2(ξ₁^{T}(\vec{p}′×\vec{q}″₃),\vec{p}′^{T}\vec{q}″₃) (11)

\vec{p}′=\vec{p}−ξ₁ξ₁^{T}\vec{p}

\vec{q}″₃=\vec{q}₃−ξ₁ξ₁^{T}\vec{q}₃

From the hardware joint limits of the robot, the range of θ₂ is −17.0°≤θ₂≤46.0°. Therefore, θ₁ and θ₂ are unique since \hat{q}₃ is unique (γ<0).

For a given target \vec{p} and θ₃, a unique solution (θ₁, θ₂)^{T} that aligns the needle on target is calculated by solving the inverse kinematics ( [symbol] ) problem as shown above:

(θ₁,θ₂)^{T}= [symbol] (\vec{p},θ₃) (12)

FIG. 2 illustrates a graphical view of an example of optimizing the approach angles for target point p=(10, 10, −100)^{T} and scan position (θ₁^{s}, θ₂^{s}=(0, 0). For example, the light grey curves in FIG. 6 show θ₁ and θ₂ as a function of θ₃ for a target p=(10, 10, −100)^{T} and scan position (θ₁^{s}, θ₂^{s})=(0, 0). The optimal approach of the TRUS probe to a target is one that minimizes the movements of the θ₁ and θ₂ from their scan positions θ₁^{s} and θ₂^{s}:

$${θ}_{3}^{opt} = {argmin}_{{θ}_{3}} [ {( {θ}_{1} - {θ}_{1}^{s} )}^{2} + {( {θ}_{2} - {θ}_{2}^{s} )}^{2} ] \qquad ( 13 )$$

For example, the dark grey curve in FIG. 6 shows the sum of squared values for all θ₃ angles, and the green line shows the optimal value.

The optimal θ₁ and θ₂ angles are:

(θ₁^{opt},θ₂^{opt})^{T}= [symbol] (\vec{p},θ₃^{opt}) (14)

A gradient descent algorithm was used to determine the minimum solution. Given the shapes of the curves, the global minimum was found by starting the minimization from each limit and the center of the θ₃ range and retaining the lowest solution.

2) Optimizing the Order of the Biopsy Cores

Once the optimal approach angles are calculated for a set of n biopsy points, the order of the biopsies can also be optimized to minimize the travel of the probe, a problem known as the travelling salesman problem (TSP). The TSP is to find the shortest route that starts from the initial scan position, visits each biopsy point once, and returns to the initial scan position \vec{s}₀=(θ₁^{s}, θ₂^{s}, 0)^{T}. The optimal approach of biopsy point i=1, . . . , n is \vec{s}ᵢ=(θ₁ⁱ, θ₂ⁱ, θ₃ⁱ)^{T}. The squared distance between a pair of points is:

d(\vec{s}ᵢ,\vec{s}ⱼ)=(\vec{s}ᵢ−\vec{s}ⱼ)^{T}(\vec{s}ᵢ−\vec{s}ⱼ) for i≠j (15)

The goal is to find an ordering π that minimizes the total distance:

$$D = {\sum }_{i = 0}^{n - 1} d ( {\vec{s}}_{π ( i )} , {\vec{s}}_{π ( i + 1 )} ) + d ( {\vec{s}}_{π ( n )} , {\vec{s}}_{π ( 0 )} ) \qquad ( 16 )$$

The solution of the TSP is found using a 2-step algorithm. FIGS. 7A and 7B show an example of n=12 biopsy points, represented in robot joint coordinates, as illustrated in FIG. 7A, and Cartesian space of the prostate, as illustrated in FIG. 7B. FIGS. 7A and 7B illustrate graphical views of examples of the location of 12 biopsy cores in joint coordinates, as illustrated in FIG. 7A and Cartesian coordinates, as illustrated in FIG. 7B. In joint coordinates, the graph is rather tall as expected, because all points are approached optimally, with small lateral motion. The line connecting the points marks the optimal order of the biopsy cores for minimal travel. Cores are then labeled accordingly, from P1 to P12.

3) Prostate Coordinate System (PCS) and Extended Sextant Biopsy Plan

The algorithms above calculate the optimal approach and order for a set of biopsy points. Systematic or targeted biopsy points can be used, depending on the procedure and decision of the urologist. For systematic biopsy, the present invention also includes software tools to help the urologist formulate the plan, graphically, based on the acquired 3D ultrasound. The most common systematic biopsy plan is the extended sextant plan of 12-cores. The plan uses a Prostate Coordinate System (PCS) that is derived based on anatomic landmarks of the prostate. The origin of the PCS is defined at the midpoint between the apex (A) and base (B) of the prostate. The direction of the PCS follows the anatomic Left-Posterior-Superior (LPS) system (same as in the Digital Imaging and Communications in Medicine (DICOM) standard). The S axis is aligned along the AB direction, and P is aligned within the sagittal plane.

FIGS. 8A-8D illustrate image views of prostate biopsy plans. FIG. 8A illustrates apex (A) and base (B) landmarks of the Prostate Coordinate System (PCS). FIG. 8B illustrates a 12-Core plan shown in LS (coronal) plane. FIG. 8C illustrates a project plan posteriorly below the urethra. FIG. 8D illustrates a sextant plan with cores shown in 3D over a coronal slice. FIG. 8A shows an example with the apex (A) and base (B) in a central sagittal view of the gland. In software, the A&B points are selected manually, and several steps allow their location to be quickly and successively refined: 1) Select A&B points in the original rotary slices (para-coronal); 2) Refine their locations in the current LP (axial) re-slices of the volume image and orient the P direction; 3) Refine the A and B in the current SL (coronal) re-slices; 4) Refine the A and B in the current PS (sagittal) re-slices. In the above, the PCS location is updated after each step.

The PCS facilitates the definition of the biopsy plan. A SB template is centered over the PCS and scaled with the AB distance. As such, defining the PCS allows to define the plan without the need for prostate segmentation. For the extended sextant plan, the 12 cores are initially placed by the software on the central coronal (SL plane) image of the gland and scaled according to the AB distance. The software then allows the physician to adjust the location of the cores as needed, as illustrated in FIG. 8B. Since prostate biopsies are normally performed more posteriorly, towards the peripheral zone (PZ) where the majority of PCa tumors are found (68%), the program switches the view to central sagittal (PS), and displays a curve that can be pulled posteriorly below the urethra, as illustrated in FIG. 8C. The 12-cores are then projected in the P direction to the level of this curve to give the final 3D biopsy plan, as illustrated in FIG. 8D.

The robot control component of the software is used to monitor and control the robot, as illustrated in FIG. 3C. A watchdog built on hardware and software removes the motor power should a faulty condition occur. FIGS. 3A-3C show an exemplary navigation screen that shows a 3D virtual environment with the robot, probe, and real-time ultrasound image. The position of all components is updated in real-time. Furthermore, the navigation screen shows the biopsy plan, the current target number and name. The names of the cores follow the clinical system (Left-Right, Apex-Mid-Base, and Medial-Lateral), and are derived automatically based on the positions of the cores relative to the PCS. The right side of the navigation screen, as illustrated in FIG. 3C, shows real time ultrasound images with an overlaid needle insertion guide. Most biopsy needles have a forward-fire sampling mechanism. The green guide marks how deep to insert the needle before firing the biopsy, so that when fired, the core is centered at the biopsy target. The depth line is located along the needle trajectory and offset from the target. The offset depends on the needle type, and is measured between the point of the loaded biopsy needle and the center of the magazine sample of the fired needle.

In an exemplary implementation of the present invention, which is not meant to be considered limiting, the TRUS probe is cleaned and disinfected as usual, mounted in the robot, and covered with a condom as usual. The patient is positioned in the left lateral decubitus position and periprostatic local anesthesia are performed as usual. With the support arm unlocked, the TRUS probe mounted in the robot is placed transrectally and adjusted to show a central sagittal view of the prostate. The support arm is locked for the duration of the procedure. The minimal level of probe insertion is adjusted under joystick control as described, herein. A 3D rotary scan is then performed under software control, as shown herein. The PCS and biopsy plan are made by the urologist. The software then optimizes the approach to each core and core order. Sequentially, the robot moves automatically to each core position. The urologist inserts the needle through the needle-guide up to the depth overlaid onto the real time ultrasound, as illustrated in FIG. 3C, and samples the biopsy manually, as usual. Ultrasound images are acquired with the needle inserted at each site for confirmation. Image acquisition is triggered automatically by the noise of the biopsy needle firing. All data, including the ultrasound images and configurations, A-B points, PCS, targets, and confirmation images are saved automatically.

Comprehensive experiments were carried out to validate the system. These experiments are included by way of example and are not meant to be considered limiting. The validation experiments include two bench tests, an imaging test, two targeting tests, and five clinical trials on patients. Needle targeting accuracy and precision results were calculated as the average and standard deviation of the needle targeting errors, respectively.

In a Robot Joint Accuracy Test, an optical tracker (Polaris, NDI, Canada) was used to measure the 3D position of a reflective marker attached to the probe (˜250 mm from RCM point) as shown in FIG. 9. FIG. 9 illustrates a perspective view of an experimental setup for robot joint accuracy test. The tracker was setup (1100 mm away from the marker) to improve the accuracy of measurement (0.078 mm).

One at a time, each joint of the robot was moved with an increment of 5° for θ₁, θ₂, θ₃, and 5 mm for τ over the entire ranges of motion. 500 position measurements of the marker were acquired and averaged at each static position.

For each axis, the measured increments between consecutive points were compared to the commanded increments. For the rotary axes, a plane was fitted to the respective point set using a least square technique. The point set was then projected onto the plane and a circle was fitted using a least square technique. Rotary axes increments were measured as the angles between the radials to each position, in plane. For the translational axis, a principal component analysis (PCA) was applied to the point set and the first principal axis was estimated. Translational axis increments were measured as the distances between consecutive points projected onto the first principal axis.

In a Robot Set Point Test, the experimental setup was similar to the previous tests, but the optical marker was fitted on a rod passed through the needle guide to simulate the needle point (˜142 mm from the RCM point, 55 mm from the probe tip). The axes were moved incrementally as follows: move θ₁ from −45° to 45° with 5° increment (19 positions); For each move θ₂ from −15° to 40° with 5° increment (12 positions); For each, move θ₃ from −90° to 90° with 30° increment (7 positions). The translation was fixed at τ=0 because its moving direction is parallel to the needle insertion axis. Each of the k=19×12×7=1596 marker locations was measured with the tracker and formed the dataset \vec{g}∈ [symbol] ³. Each commanded joint position was passed through the forward kinematics of the robot to calculate the robot-space commanded dataset \vec{h}∈ [symbol] ³. The homogeneous transformation matrix F∈ [symbol] ^{4×4} between the tracker and robot coordinates was estimated with a rigid point cloud registration technique. The virtual needle point positioning error eᵥ was evaluated as the average positioning error:

$${e}_{v} = \frac{1}{k} {\sum }_{i = 1}^{k} ‖ F \cdot {\vec{g}}_{i} - {\vec{h}}_{i} ‖ \qquad ( 17 )$$

FIGS. 10A-10C illustrate the 3D Imaging Geometric Accuracy Test and the Grid Targeting Test. FIG. 10A illustrates a setup for a grid of strings in a water tank experiments, FIG. 10B illustrates a 3D image, and FIG. 10C illustrates error estimation (>1.0 mm). In a 3D Imaging Geometric Accuracy Test, a 5-by-5 grid of strings (Ø0.4 mm) spaced 10 mm apart was built, submersed in a water tank, and imaged with a 3D rotary scan, as illustrated in FIG. 10A. The 25 grid crossing points were selected in the 3D image and registered to a grid model (same spacing) using a Horn's method. Errors between the sets were calculated and averaged. The test was repeated 5 times for different depth settings of the ultrasound machine (50, 65, 85, 110, 125 mm).

In a Grid Targeting Test, the grid described above was also targeted with the needle point to observe by inspection how close the needle point can target the crossings, as illustrated in FIG. 10B. The stylet of an 18 Ga needle (stylet diameter ˜1 mm) was inserted through the automatically oriented needle-guide and advanced to the indicated depth. No adjustments were made. Targeting errors were estimated visually to be ≤0.5 mm if the point of the needle was on the crossing, ≤1.0 mm if the error appeared smaller than the stylet diameter, and >1 mm otherwise, as illustrated in FIG. 10C. The test was repeated 3 times for grid depths of 20, 40, and 60 mm.

In a Prostate Mockup Targeting Test, a prostate mockup (M053, CIRS Inc., Norfolk, Va.) was used, as illustrated in FIGS. 11A and 11B. FIGS. 11A and 11B illustrate a targeting experiment with prostate mock-up. FIG. 11A illustrates an image view of an experimental setup, and FIG. 11B illustrates a resultant 2D displacement/deformation. The experiment followed the clinical procedure method of 12-core biopsy describe in above. The biopsy needle was an 18 Ga, 20 cm long, 22 mm throw MC1820 (Bard Medical, Covington, Ga.). In addition, the prostate was also manually segmented, and a 3D prostate surface model was generated to quantify the magnitude of interventional prostate deformations, if present. A confirmation ultrasound image was saved at each needle insertion. A post-biopsy 3D rotary scan at the initial scan location (θ₁^{s}, θ₂^{s}) was also performed for initial/final prostate shape/location comparison.

FIGS. 12A and 12B illustrate schematic diagrams of prostate displacement and prostate deformation measurements, respectively. In the analysis, the pre-acquired 3D prostate surface was intersected with the plane of the saved confirmation image to render the pre-acquired 2D prostate shape, as shown in FIG. 12A. This was then compared with the imaged prostate shape to determine the level of prostate displacement dₚ (distance between centers, \vec{c}₁, \vec{c}₂) and deformation d_{ƒ}. To measure deformations, the pre-acquired contour was translated with \vec{c}₂−\vec{c}₁ to a common center. Deformations d_{ƒ} were measured radially (φ=) 15° between the contours, as shown in FIG. 12B, and averaged for each confirmation image.

Needle insertion errors eₙ were measured as distances between the imaged needle axis and the target point, as illustrated in FIG. 15A. Overall targeting errors eₜ were calculated as the sum of the needle insertion error and the 2D displacements of the prostate dₚ.

Finally, the 3D displacement and deformation of the prostate were measured between the pre- and post-biopsy ultrasound volumes. The displacement Dₚ was the distance between the centroids of the two surfaces. Then, the pre-biopsy surface was translated to align the centers, and the deformations were calculated as a mean D_{ƒ} and maximum value D_{ƒ}^{max} of the distances between the corresponding closest points of the surfaces, as illustrated in FIG. 15B.

A final experiment was performed to visually observe the motion of the TRUS probe about the prostate and how the probe deforms the prostate. The prostate mockup was made of a soft-boiled chicken egg, peeled shell, and placed on 4 vertical poles support. The support was made to gently hold the egg so that the egg could be easily unbalanced and pushed off, to see if biopsy can be performed on the egg without dropping it. A limitation of this experiment is that the egg mockup is unrealistic in many respects. This is a way to visualize the motion of the probe about the prostate, motion that is calculated by algorithms, and is difficult to observe with closed, more realistic mockups.

In an exemplary clinical trial, that is not meant to be considered limiting, the safety and feasibility of robotic prostate biopsy was assessed. The study was carried out on five men with an elevated PSA level (≥4 ng/ml) and/or abnormal DRE. For all the cases, extended sextant systematic prostate biopsies were performed based on the protocol described herein. FIG. 13 illustrates a side view of a robotic prostate biopsy. As illustrated in FIG. 13, the robot handles the TRUS probe and the urologist handles the needle. FIG. 13 shows the system setup for the clinical trial. Needle insertion errors eₙ were calculated as described in Sec. F5. Needle targeting accuracy and precision were calculated as the average respectively standard deviation of the errors, as usual. Partial and overall procedure times were also recorded.

The joint accuracies and precision of the robot are shown in TABLE I.

TABLE I
ROBOT JOINT ACCURACY TEST RESULTS

| Joint | Accuracy | Precision |
|---|---|---|
| θ₁ [°] | 0.112 | 0.079 |
| θ₂ [°] | 0.021 | 0.028 |
| θ₃ [°] | 0.040 | 0.033 |
| τ [mm] | 0.015 | 0.013 |

FIG. 14 shows an example of the set point test results (θ₃=0°). The virtual needle point positioning error eᵥ was 0.56±0.30 mm. The maximum error was 1.47 mm. FIG. 14 illustrates a graphical view of the Robot set point test results (θ₃=0°).

The accuracies and precisions of the 25 grid points with 5 different depth settings are presented in TABLE II.

TABLE II
3D IMAGING GEOMETRIC ACCURACY TEST RESULTS

| Depth Setting d [mm] | Accuracy [mm] | Precision [mm] |
|---|---|---|
| 50 | 0.48 | 0.26 |
| 65 | 0.51 | 0.20 |
| 85 | 0.47 | 0.19 |
| 110 | 0.51 | 0.27 |
| 125 | 0.44 | 0.23 |
| Total | 0.48 | 0.23 |

For the grid depth of 20 mm, the number of experiments with targeting errors ≤0.5, ≤1.0, and >1.0 mm were 18, 6, and 1 respectively. For the grid depth of 40 mm, the corresponding number were 21, 3, and 1, respectively. For the grid depth of 60 mm, the corresponding numbers were 20, 5, and 0. The two cases when the errors were >1.0 mm appeared to be ≤1.5 mm. One of these cases is shown in FIG. 10C.

FIGS. 15A and 15B illustrate image views of targeting results with prostate mock-up. FIG. 15A illustrates needle insertion error, and FIG. 15B illustrates 3D prostate deformation. FIGS. 15A and 15B show the needle insertion error and the 3D distance map of the prostate deformation. The 3D displacement Dₚ and deformation D_{ƒ} of the prostate were 0.58 and 0.20 mm, respectively. The maximum deformation distance D_{ƒ}^{max} was 0.89 mm.

TABLE III
PROSTATE MOCKUP TARGETING TEST RESULTS

| | | |
|---|---|---|
| Target | Target | Errors [mm] |

| No. | Position | dₚ | d_{f} | eₙ | eₜ |
|---|---|---|---|---|---|
| 1 | RAM | 1.21 | 0.71 | 0.79 | 2.01 |
| 2 | RAL | 0.38 | 0.46 | 0.41 | 0.79 |
| 3 | RML | 0.48 | 0.30 | 0.60 | 1.09 |
| 4 | RBL | 0.26 | 0.41 | 0.70 | 0.96 |
| 5 | RBM | 1.13 | 0.37 | 0.80 | 1.93 |
| 6 | RMM | 0.88 | 0.45 | 0.70 | 1.58 |
| 7 | LBL | 0.77 | 0.39 | 0.40 | 1.17 |
| 8 | LML | 1.03 | 0.71 | 0.31 | 1.35 |
| 9 | LAL | 0.21 | 0.57 | 0.69 | 0.91 |
| 10 | LBM | 0.76 | 0.42 | 0.60 | 1.36 |
| 11 | LMM | 0.97 | 0.41 | 0.51 | 1.48 |
| 12 | LAM | 1.26 | 0.36 | 0.31 | 1.57 |

| | | | | |
|---|---|---|---|---|
| Max | 1.26 | 0.71 | 0.80 | 2.01 |
| Accuracy | 0.78 | 0.46 | 0.57 | 1.35 |
| Precision | 0.37 | 0.13 | 0.18 | 0.39 |

The biopsy on the egg experiment performed the 3D scan and positioned the probe for biopsy without pushing the egg off the support.

The robot allowed 3D imaging of the prostate, 3D size measurements, and volume estimation. The results are presented in TABLE IV.

TABLE IV
PROSTATE SIZE AND VOLUME

| | | | | Prostate |
|---|---|---|---|---|
| | Prostate Size [mm] | | | Volume |
| Patient | Superior-Inferior | Anterior-Posterior | Left-Right | [cm³] |
| 1 | 38.85 | 30.32 | 49.27 | 28.45 |
| 2 | 57.47 | 46.18 | 64.33 | 85.55 |
| 3 | 48.33 | 31.63 | 44.96 | 44.82 |
| 4 | 52.78 | 40.45 | 69.44 | 83.94 |
| 5 | 50.81 | 43.85 | 56.68 | 75.70 |

The robot also enabled hands-free TRUS operation for prostate biopsy and all 5 procedures were successful from the first attempt. The biopsy procedures took 13 min on average. Slight patient motion at the time of biopsy firing was occasionally observed. No remnant prostate shift was observed. There were no adverse effects due to the robotic system. Three of the five patients had malignant tumor with biopsy Gleason Scores of 3+3, 3+4, and 3+3. Numerical results are presented in Table V.

TABLE V
CLINICAL TRIAL RESULTS

| No of 3D scan ultrasound slices | | 238 | |
|---|---|---|---|
| Average time | 3D image scan | 0.48 | min |
| | PCS and biopsy plan | 6.26 | min |
| | Biopsy sampling | 4.42 | min |
| | Total procedure | 13.02 | min |
| Needle Targeting* | Accuracy | 0.51 | mm |
| | Precision | 0.17 | mm |
| Cancer diagnosis | | 3/5 | patients |

*Over 4 patients (missed recording all confirmation images on a patient)

Image registration is a commonly required step of clinical procedures that are guided by medical images. This step must normally be performed during the procedure and adds to the overall time. With the TRUS robot, and also with fusion biopsy devices, intra-procedural registration is not required. Instead, a calibration is performed only once for a given probe. The probe adapter was designed to mount it repeatedly at the same position when removed for cleaning and reinstalled, to preserve the calibration.

Bench positioning tests show that the robot itself can point a needle with submillimeter accuracy and precision. The geometric accuracy and precision of 3D imaging were submillimetric. Combined, image-guided targeting errors in a water tank (no deformations) were submillimetric in 97.3% of the tests and <1.5 mm overall. Experiments on prostate mockups showed that changes in the position and deformation of the prostate at the time of the initial scan and biopsy were submillimetric. Overall, needle targeting accuracy in a deformable model was 1.43 mm. The biopsy on the egg experiment showed that the robot can operate the TRUS probe gently, with minimal pressure.

Preserving small prostate deformations at the time of the 3D scan and biopsy was achieved by using primarily rotary motion about the axis of the probe and minimizing lateral motion. A similar approach may be intuitively made with the Artemis (Eigen) system, which uses a passive support of the arm of the TRUS probe. Here, the optimal approach angles are derived mathematically.

In the experiments optimal solutions were uncommon, unintuitive, and not ergonomic to freehand. FIG. 16A shows the way that a physician would normally freehand the probe to a site. Instead, shows the optimal approach to the same site, which is not ergonomic and difficult to freehand. Freehand biopsy is often suboptimal, because turning the probe upside down is not ergonomic. FIGS. 16A and 16B illustrate image views of an example of free handing the probe to a site. FIG. 16A illustrates common handing the probe to a site, and FIG. 16B illustrates an optimal handing the probe to a site.

A coordinate system associated with the prostate (PCS), and a method to formulate a SB plan based on the PCS are also included in the present invention. Several prostate biopsy systems use intraoperative methods to locate a system that is similar to the PCS, by manually positioning the probe centrally to the prostate. In the approach of the present invention, the PCS is derived in the 3D image, possibly making it more reliable. The two methods were not compared in the present report.

At biopsy, images of the inserted needle are commonly acquired after firing the needle. At hands-free biopsy or with other biopsy devices, the acquisition is triggered by the urologist, from a button or pedal. Herein, a simple innovation is presented that triggers the acquisition automatically, by using a small microphone circuit located next to the needle that listens for the firing noise that biopsy needles commonly make, and triggers the acquisition immediately after the biopsy noise. Capturing the image at the exact moment increases precision and reliability. The automation simplifies the task for the urologist, avoids forgetting to capture the image, and makes the process slightly faster.

The results of the clinical trial show that robot-assisted prostate biopsy was safe and feasible. Needle targeting accuracy was on the order of 1 mm. Additional possible errors such as errors caused by patient motion should be further evaluated and minimized. No significant patient movement was observed during the limited initial trial, and no loss of ultrasound coupling was experienced. The development of a leg support to help the patient maintain the position and additional algorithms to correct for motion are in progress.

The TRUS robot and the Artemis device are the only systems that manipulate the probe about a RCM fulcrum point. With the other systems that freehand the probe, the fulcrum is floating. Thus far, there has not been patient discomfort related to fixing the fulcrum. Performing biopsy with minimal probe pressure and motion could ease the discomfort and help the patient to hold still.

Clinically, the robot of the present invention is for transrectal biopsy and the other approach is transperineal. Traditionally, transperineal biopsy was uncommon because requires higher anesthesia and an operating room setting, but offered the advantage of lower infection rates. New transperineal approaches for SB and cognitive TB are emerging with less anesthesia and at the clinic. Yet, the mainstream prostate biopsy is transrectal. Several methods reported herein, such as the PCS and TRUS imaging with reduced prostate deformations could apply as well to transperineal biopsy. The robot of the present invention can guide a biopsy needle on target regardless of human skills. The approach enables prostate biopsy with minimal pressure over the prostate and small prostate deformations, which can help to improve the accuracy of needle targeting according to the biopsy plan.

It should be noted that the software associated with the present invention is programmed onto a non-transitory computer readable medium that can be read and executed by any of the computing devices mentioned in this application. The non-transitory computer readable medium can take any suitable form known to one of skill in the art. The non-transitory computer readable medium is understood to be any article of manufacture readable by a computer. Such non-transitory computer readable media includes, but is not limited to, magnetic media, such as floppy disk, flexible disk, hard disk, reel-to-reel tape, cartridge tape, cassette tapes or cards, optical media such as CD-ROM, DVD, Blu-ray, writable compact discs, magneto-optical media in disc, tape, or card form, and paper media such as punch cards or paper tape. Alternately, the program for executing the method and algorithms of the present invention can reside on a remote server or other networked device. Any databases associated with the present invention can be housed on a central computing device, server(s), in cloud storage, or any other suitable means known to or conceivable by one of skill in the art. All of the information associated with the application is transmitted either wired or wirelessly over a network, via the internet, cellular telephone network, RFID, or any other suitable data transmission means known to or conceivable by one of skill in the art.

Although the present invention has been described in connection with preferred embodiments thereof, it will be appreciated by those skilled in the art that additions, deletions, modifications, and substitutions not specifically described may be made without departing from the spirit and scope of the invention as defined in the appended claims.

1. A system for prostate biopsy comprising:
a robot-operated, hands-free, ultrasound probe and manipulation arm;
a biopsy needle;
a robot controller, wherein the robot controller is configured to communicate with and control the manipulation arm and ultrasound probe in a manner that minimizes prostate deflection; and
an ultrasound module for viewing images from the ultrasound probe.

2. The system of claim 1 further comprising the robot controller being programmed with a prostate coordinate system.

3. The system of claim 2 wherein the prostate coordinate system comprises a program for
determining the prostate coordinate system based on anatomical landmarks of a prostate.

4. The system of claim 3, where the anatomical landmarks are the apex (A) and base (B) of the prostate;
and the program for determining the prostate coordinate system further includes using A and B to determine a prostate coordinate system (PCS) for the prostate; and
determining the direction of the PCS based on the Left-Posterior-Superior (LPS) system, wherein an S axis is aligned along the AB direction and P is aligned with a saggital plane.

5. The system of claim of 1 further comprising, calculating an optimal approach and order for a set of biopsy points determined from the PCS.

6. The system of claim 1 further comprising the robot controller being programmed with a systematic or targeted biopsy plan.

7. The system of claim 1 wherein the robot controller allows for computer control of the ultrasound probe and manipulation arm.

8. The system of claim 1 wherein the robot controller allows for physician control of the ultrasound probe and manipulation arm.

9. The system of claim 1 wherein the manipulation arm moves the probe with 4-degrees-of-freedom.

10. The system of claim 1 further comprising a microphone, wherein the microphone triggers automatic acquisition of ultrasound images based on firing noise or a signal from the biopsy needle.

11. The system of claim 1 wherein the ultrasound probe is configured to apply minimal pressure over a prostate gland to avoid prostate deformations and skewed imaging.

12. The system of claim 11 wherein the prostate can be approached with minimal pressure and deformations also for biopsy.

13. The system of claim 10 further comprising automatically acquiring images from medical imaging equipment based on firing noise of a biopsy needle, or signal from another medical instrument.

14. The system of claim 11 wherein the images are acquired for a purpose of documenting a clinical measure.

15. A method for biopsy of a prostate comprising:
determining a midpoint between an apex (A) and base (B) of the prostate;
using A and B to determine a prostate coordinate system (PCS) for the prostate;
determining the direction of the PCS based on the Left-Posterior-Superior (LPS) system, wherein an S axis is aligned along the AB direction and P is aligned with a saggital plane;
calculating an optimal approach and order for a set of biopsy points determined from the PCS.

15. The method of claim 12 further comprising imaging the prostate with an ultrasound probe with minimal pressure over a prostate gland to avoid prostate deformations and skewed imaging.

17. The method of claim 14 wherein the prostate can be approached with minimal pressure and deformations also for biopsy.

18. The method of claim 13 further comprising automatically acquiring images from medical imaging equipment based on firing noise of a biopsy needle, or signal from another medical instrument.

19. The method of claim 13 further comprising acquiring the images for a purpose of documenting a clinical measure.

20. The method of claim 13 further comprising triggering automatic acquisition of ultrasound images based on firing noise or a signal from the biopsy needle acquired by a microphone.

21. The method of claim 14 further comprising computer control of the ultrasound probe and manipulation arm.

22. The method of claim 19 wherein the computer control allows for physician control of the ultrasound probe and manipulation arm.
